(19) United States
(12) Patent Application Publication
(10) Pub. No.: US 2025/0288631 A1
(43) Pub. Date: Sep. 18, 2025

(54) PHYTOTHERAPY FORMULATION FOR SEXUAL DYSFUNCTIONS

(71) Applicants: Valeria DOS SANTOS COZZOLINO YUGUE, COTIA (BR); Adriana SCHULZ DOS SANTOS, ATIBAIA (BR)

(72) Inventors: Valeria DOS SANTOS COZZOLINO YUGUE, COTIA (BR); Adriana SCHULZ DOS SANTOS, ATIBAIA (BR)

(21) Appl. No.: 18/860,123

(22) PCT Filed: Apr. 13, 2023
(86) PCT No.: PCT/BR2023/050117

§ 371 Date: Oct. 25, 2024

(30) Foreign Application Priority Data
Apr. 26, 2022 (BR) 1020220078971

(51) Int. Cl. A61K 36/28; A61K 9/00; A61K 47/12; A61K 47/18; A61K 47/38

(52) U.S. Cl. CPC A61K 36/28; A61K 9/0031; A61K 9/0034; A61K 47/12; A61K 47/183; A61K 47/38

(57) ABSTRACT

A phytotherapy formulation for preventing or treating sexual dysfunctions, and more specifically, a formulation containing standardized extract of Spilanthes acmella, is provided which is useful as an adjuvant in the prevention and treatment of female and male sexual dysfunctions, including hypoactive sexual desire disorder (HSDD).

### FIELD OF THE INVENTION

This is an invention in the field of medicines, cosmetics, and health products. Specifically, the present invention relates to a herbal formulation containing standardized extract of Spilanthes acmella, useful as an adjuvant in the prevention and treatment of female and male sexual dysfunctions, including hypoactive sexual desire disorder (HSDD).

### FUNDAMENTALS OF THE INVENTION

Jambu (Spilanthes acmella var oleracea) is native to Brazil and belongs to the Asteraceae family. This species is particularly rich in alkylamides and N-isobutylamides, and spilanthol is considered responsible for the biological activities of the species. This plant is widely used in northern Brazilian cuisine. It is also traditionally used in some regions of Brazil as a powerful aphrodisiac to help with a lack of sexual desire.

Jambu extract usually has a dark brown color, which is an obstacle to the purpose of the invention, considering that the supplier can add titanium dioxide to it, a pigment with high bleaching power, to obtain a light-colored extract. However, bibliographic research has shown a strong tendency to withdraw titanium dioxide pigment from products for human consumption (EFSA, Titanium dioxide: E171 no longer considered safe when used as a food additive, 2021).

Every standardized plant extract must have markers to indicate the quality standard. These markers can be active or just analytical markers, according to RDC 26/2014 (Brazilian Health Regulatory Agency—Anvisa, 2014). Jambu extract's marker is spilanthol, which is an active marker.

The vagina is a microenvironment sensitive to pH variations and to any substances that may affect its physiological balance (Machado, Preclinical performance of vaginal semisolid products: technological and safety evaluations assuming physiologic parameters. Thesis for Doctoral Degree in Pharmaceutical Sciences. Universidade da Beira Interior, Portugal, 2018).

In a research carried out on existing vaginal products and their compositions, the extensive use of allergenic preservatives was noted, such as: the class of parabens, mainly propylparaben and methylparaben; the use of alcohols such as glycerin and propylene glycol, which dehydrate the vaginal mucosa; the use of lipids that can cause occlusions; or surface-active agents that denature protein structures, affecting the normal vaginal flora and causing infections.

Likewise, for the development of a formulation for vaginal and anal use, physical aspects such as appearance, viscosity, consistency, and hydrophilicity were considered, in addition to chemical aspects such as pH, maintenance of active content, low formation of degradation products, and microbiological preservation. Inputs compatible with spilanthol and with the physiological characteristics were chosen, aiming at the physical-chemical stability of the active, the intended performance, and the comfort when using the product.

Some products on the market contain jambu, but they also contain allergenic substances in their composition, and most are regulated by ANVISA as cosmetic products, exempt from registration.

In the state of the art, some patent documents containing jambu derivatives in the claimed compositions were found. However, none of them presented a herbal formulation containing a standardized extract of jambu, for topical use, containing a maximum dose of spilanthol of 0.2% (2 mg/g) with low permeation in the mucosa (approximately 0.32% of the initial dose). The proposed formulation in this invention has low permeation on the mucosa, which is extremely relevant to avoid side effects resulting from excessive dosage of spilanthol in the genital and anal regions. The dosage used in the present invention allows for limited permeability precisely so as not to cause irritation to the mucosa.

Patent application BR0100254-6 claims a sensitizing composition containing jambu in the form of oily resin, indicated as a deodorant, antiperspirant, moisturizer, or ointment, without any mention of indications for the prevention or treatment of diseases, or for the relief of symptoms of sexual dysfunctions.

The patent application BR1004308-0 claims the production process of the hydroethanolic extract of Spilanthes acmella. However, the hydroethanolic extract of S. acmella would not be suitable for vaginal use, due to the presence of ethanol. Additionally, it has not been standardized on Spilanthol.

Patent application BR102019007235-0 claims an anesthetic composition based on Acmella sp. for the vaginal introitus, with the function of reducing sensitivity in the vagina, relieving pain during sexual intercourse. For the development of this product, the mechanisms of action of jambu extract were not studied, mainly the fact that jambu increases blood flow in the genital region. Additionally, patent application BR102019007235-0 claims a composition based on aqueous extract of Acmella, with Natrozol®, vegetable glycerin, and propylene glycol. It is interesting to point out that the claimed formulation is composed of 60% aqueous extract of Acmella, which causes unwanted side effects, as previously described. Additionally, glycerin and propylene glycol cause an imbalance in the vaginal flora.

The dosage used in the present invention allows limited permeability and does not cause mucosal irritations or other adverse effects related to the mechanism of action of the Spilanthes acmella var oleracea extract.

### BRIEF DESCRIPTION OF THE DRAWINGS

FIG. 1 presents a flowchart of the processes for manufacturing the bench gel pilot batches;

FIGS. 2 and 3 show pictures of the jambu gel;

FIG. 4 shows the molecular formula of spilanthol;

FIG. 5 shows predominant chemical forms of Spilanthol versus different pH ranges;

FIG. 6 shows the inside of the aluminum tube with gold varnish as an internal coating and with Darex;

FIG. 7 presents a photo of aluminum tubes with gold varnish as an internal coating and with Darex, these tubes being filled with jambu gel;

FIG. 8 presents a photo of jambu gel manufacturing in the planetary mixer (internal part of the equipment) with the mixing rod (elevated);

FIG. 9 presents a photo of jambu gel manufacturing in the planetary mixer (internal part of the equipment) without the mixing rod;

FIG. 10 presents a photo of the planetary mixer (external part of the equipment);

FIG. 11 shows the phytochemical profile of the jambu gel sample. Caption: 1.Jambu gel; 2. Jambu mold extract; 3. Alpha Amyrin pattern; 4. Beta Sitosterol pattern; 5. Methanol (diluent); 6. Jambu gel placebo;

FIG. 12 shows the HPLC chromatographic profile for the Spilanthol Standard;

FIG. 13 shows the HPLC chromatographic profile for Jambu Extract;

FIG. 14 shows the HPLC chromatographic profile for Jambu Gel;

FIG. 15 presents results of the analytical validation of the Spilanthol content methodology in Jambu Gel—part 1. Source: DALL. Reproduction of the Analytical Methodology Validation Report for Determination of Spilanthol Content in Jambu Gel by HPLC —REV.MET.323—Version 00;

FIG. 16 presents results of the analytical validation of the Spilanthol content methodology in Jambu Gel—part 2. Source: DALL. Reproduction of the Analytical Methodology Validation Report for Determination of Spilanthol Content in Jambu Gel by HPLC —REV.MET.323—Version 00;

FIG. 17 presents results of the analytical validation of the methodology for Spilanthol content in Jambu Gel—part 3. Source: DALL. Reproduction of the Analytical Methodology Validation Report for Determination of Spilanthol Content in Jambu Gel by HPLC—REV.MET.323—Version 00.

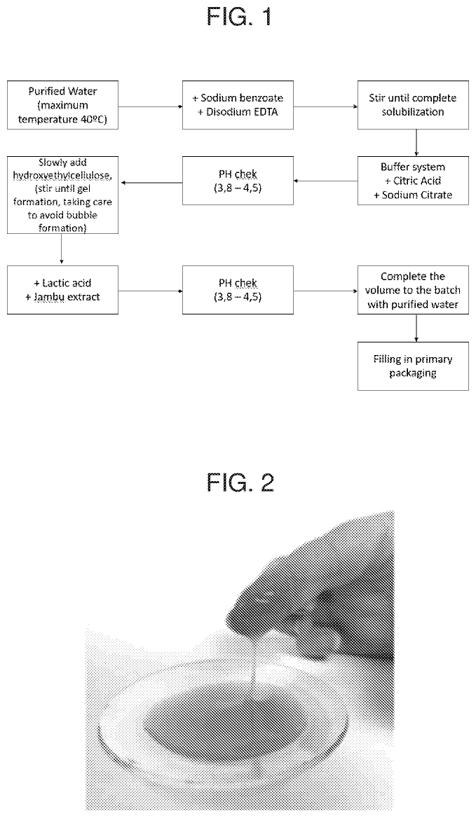

### DESCRIPTION OF THE INVENTION

This invention relates to a herbal formulation containing standardized extract of Spilanthes acmella, useful as an adjuvant in the prevention and treatment of female and male sexual dysfunctions, including hypoactive sexual desire disorder (HSDD).

The extract is produced with water and ethanol extractor solvents, then concentrated using polysorbate as an excipient, and has spilanthol as a marker at a concentration of at least 10%.

Jambu has been found to contribute to arousal, which is the phase of increased parasympathetic blood flow to the genitals that, in women, leads to clitoral erection and helps producing vaginal lubrication. As for anal intercourse, jambu can contribute to reducing pain by relaxing the muscles. This mechanism of action of jambu extract occurs due to the involvement of nitric oxide (NO), which leads to relaxation of smooth muscles, facilitating the entry of blood into the treated region.

The product was developed according to the guidelines of the ICH Q8 guide (ICH, Pharmaceutical development Q8(R2), 2009) in order to obtain a pharmaceutical product similar to a lubricant containing a standardized active ingredient, seeking to be the first product correctly regulated in the category recommended by Anvisa. It will be the first with clinical evidence of the effect of spilanthol as an adjuvant in the treatment of sexual dysfunctions, such as TDSH, which has a high incidence in the female population world-wide. The product can also be used to relieve pain during vaginal or anal intercourse.

Bibliographical research was carried out on the physicochemical and microbiological degradation kinetics of the spilanthol molecule and the future pharmaceutical form, describing any and all potential sensitivity and incompatibility present. A Risk Analysis Report for the product was prepared, which marked the initial list of inputs (excipients) and primary packaging materials that were evaluated according to the defined kinetics. The first formulation (Prototype 1) of the product was proposed, and then Prototype 2 based on necessary adjustments.

Based on the test methodology for spilanthol in the standardized jambu extract, initial tests were carried out on prototypes of the product formulation containing jambu extract, generating the definitive methodology that was subsequently tested, optimized, and validated.

From the risk analysis, the excipients were defined for the manufacture of bench prototype batches, confirming the base composition of the formulation. There was an adjustment in relation to the initial objective of the project in view of the controversial publications and the chance of banning Titanium Dioxide as a pigment and whitening agent, as revealed in bibliographic research.

The batches manufactured in bench scale showed satisfactory characteristics for the quality standards. The batches were evaluated with the test methodology already adjusted and optimized.

Challenge tests were carried out with bench scale batches, simulating conditions of use (oral and not vaginal simulations were made), and physical and chemical stress, in order to mimic events prior to stability studies in compliance with standards. Example: temperature cycles of 60° C. and fridge for up to 10 days; stereoscopic analyzes to visualize potential crystallizations; chemical analyzes of spilanthol content, pH, sensorial analyzes and other applicable ones, according to ICH Q8 (ICH, Pharmaceutical development Q8(R2), 2009).

The validity of the analytical methodology was carried out after studying the performance of the methodology with the bench batches and making the final adjustments. The methodology was validated according to Anvisa Guide RDC 166/2017 (Anvisa, 2017).

With regard to the analytical methods employed, once developed, their confirmation through validation occurred in accordance with the requirements of Anvisa, RDC 166/2017 (Anvisa, 2017). All methodology was developed and validated by an analytical laboratory accredited for quality control analyzes for future registration submission, in other words, the laboratory is a member of the Rede Brasileira de Laboratórios Analíticos em Saúde (Reblas), which is composed of analytical laboratories authorized by Anvisa as per RDC No. 390/2020 (Anvisa, 2020).

Four analytical methodologies were developed and validated for the active input and for the finished product: a. identification by thin layer chromatography (TLC) in the extract; and b. in the finished product; c. determination of spilanthol in the plant extract of jambu, by high performance liquid chromatography (HPLC); and determination of spilanthol in the developed product, jambu gel, by high performance liquid chromatography (HPLC).

The accelerated stability study is designed to evaluate possible physical, chemical, and microbiological alterations in drugs, under forced storage conditions, in order to help determine the shelf life of the drug and to evaluate the effect of short excursions outside the recommended conservation care for the product. The accelerated study only provides an idea of the behavior of the product, which should be confirmed through the long-term study, RDC 318/2019 (Anvisa, 2019).

The long-term stability study is designed to verify the physical, chemical, and microbiological characteristics of the medicine, under storage conditions (which will be expressed on the product labeling), and the proposed expiry date. The expiration date must be determined and confirmed by a long-term stability study in accordance with RDC 318/2019 (Anvisa, 2019).

The jambu extract that complies with quality standards is produced from aerial parts of the jambu species Spilanthes acmella var oleracea, using water and ethanol as extracting solvents. In its manufacturing process, after the main extraction stage, the extract is concentrated using polysorbate 80 as the main excipient, which is an excipient approved for use in medicines.

Due to products containing titanium dioxide being forbidden, we decided to use the original plant extract without aesthetic additives, managing the risk of having to remove the pigment in the future due to some new regulatory requirement by Anvisa, modifying the qualitative composition of the product, and impacting on the studies yet to be done. Concomitantly, it was observed that products with a more natural appearance became a trend, initially in international markets and currently also in Brazil, without dyes or other additives, as an opportunity to update the product with marketing objectives.

The standardized concentration of spilanthol in the extract is at least 10% in weight, which is considered a high concentration for plant extracts, but which, on the other hand, allows the use of a small amount of the extract in the final product, and the reduction of the impact of the original color of the raw material on the color of the finished product.

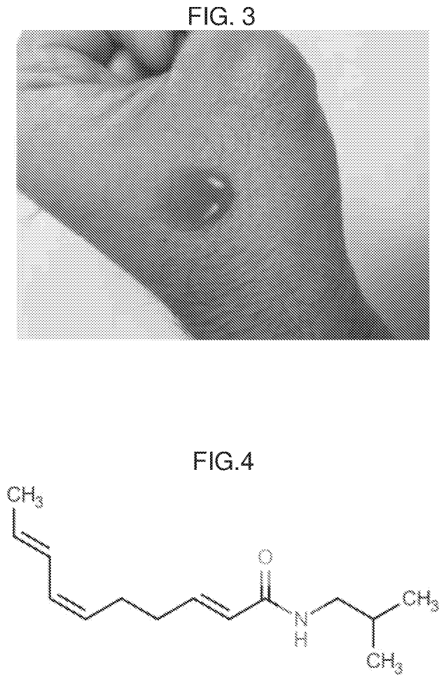

Tests were carried out aiming to define the organoleptic characteristics of the gel. The obtained color was closer to green, not brown anymore, a fact that would meet the modern trends of “more natural” looking, plus with the non-use of titanium dioxide as a marketing advantage. Through FIGS. 2 and 3 we can observe the pleasant appearance of the gel, moss green in color, and when in contact with the skin, it has a translucent appearance.

The first stage of development consisted of preparing a risk analysis of the asset's quality versus a formulation propose and industrial process, within the concepts of Quality by Design. According to the ICH guide Q8 (ICH, Pharmaceutical development Q8(R2), 2009), quality risk management must be applied throughout the life cycle to define and control the critical attributes of quality, which must be correlated to critical process parameters to provide robustness to industrial processes through the control of their critical variables.

In the theoretical pre-formulation study, the degradation kinetics of spilanthol was evaluated in order to begin the selection of excipients compatible physically and chemically with the active, which would provide suitability for use and be elements of control of risks of degradation during the shelf life of the product, that is, within the shelf life initially estimated at 24 months, which is a technically viable shelf life that meets logistical and commercial demands.

The spilanthol molecule has an amide group that presents potential for degradation by hydrolysis and subsequent oxidation. It is important that hydrolysis is managed so that oxidation is minimized or inhibited through a proper choice of formulation components. This information was important for the beginning of the pre-formulation, when the characteristics of the projected product begin to be outlined with the search for the best excipients to be used.

Hydrophilic formulations are more suitable for the vaginal route of administration (Machado et al., Vaginal semisolid products: Technological performance considering physiologic parameters. European Journal of Pharmaceutical Sciences, 2017), but due to the presence of water they may favor spilanthol hydrolysis reactions. Hydrolysis reactions occur in specific pH ranges for each active.

To define the specification range for the pH of the product, the physiological vaginal pH was also considered and an optimal range for the product, both for the stability of the product's active, and for the maintenance of the homeostatic balance of the route of drug administration. Lactic acid was chosen as an auxiliary pH excipient because it is a weak and physiologically compatible acid, in addition to being a metabolism product present in the normal flora of the vagina (Machado, Preclinical performance of vaginal semisolid products: technological and safety evaluations assuming physiologic parameters. Thesis for Doctoral Degree in Pharmaceutical Sciences. Universidade da Beira Interior, Portugal, 2018).

Tests were made to analyze the critical attributes of the product's quality. The results can be seen in Table 1, below.

TABLE 1

| Critical Quality Attributes | | |
|---|---|---|
| (CQA) | Justification | Critical process parameters |
| Aspect | Color changes may occur | Temperature and Waiting |
| | due to oxidative processes; | time between steps |
| | changes in consistency and | |
| | viscosity may occur due to | |
| | microbiological contamination | |
| pH | Changes may occur due to | PH range, through buffering |
| | chemical reactions by | |
| | hydrolysis and formation of | |
| | degradation products | |
| Spilanthol content | Active substance whose | Mixing Time, Mixing Speed, |
| | chemical integrity is related | and Temperature |
| | to the effectiveness of the | |
| | product | |

An important physical aspect is the viscosity and consistency of the product for vaginal application, which should mimic as closely as possible the characteristics of the normal mucus of a healthy woman. The viscosity agent hydroxyethylcellulose (commercial name Natrosol®) was chosen due to its hydrophilic characteristics, non-ionic character (not influenced by pH), and compatibility with physiological pH. The gel formed with hydroxyethylcellulose presented an appearance similar to vaginal mucus at the end of the bench tests to define concentration.

Tests were carried out to define the ideal concentration versus the desired consistency for use, application, and dispensing (exit from the proposed packaging). The results are shown in Table 2, below.

TABLE 2

| Tests Hydroxyethylcellulose | Results |
|---|---|
| concentration (%) | (Visual and sensory methods) |
| 0.5 | Very fluid and may leak during use |
| 1.0 | Very fluid and may leak during use |
| 2.0 | Fluid, suitable for application, but |
| | inaccurate dispensing |
| 2.5 | Adequate consistency, satisfactory for |
| | application and dispensing |

The base manipulated on a bench with 2.5% hydroxyethylcellulose was subjected to a primary stability study (without the presence of plant extract), which consists of carrying out exploratory tests of submission to short-term stress to assess potential physical weaknesses, ICH Q1A (ICH, 2003). The sample was subjected to cycles of 12 hours at low temperature (refrigerator at 5° C.) and 12 hours at high temperature (stove at 60° C.), for 10 days. The base tested did not show changes in appearance or break in consistency, demonstrating safety for follow-up to the next phases.

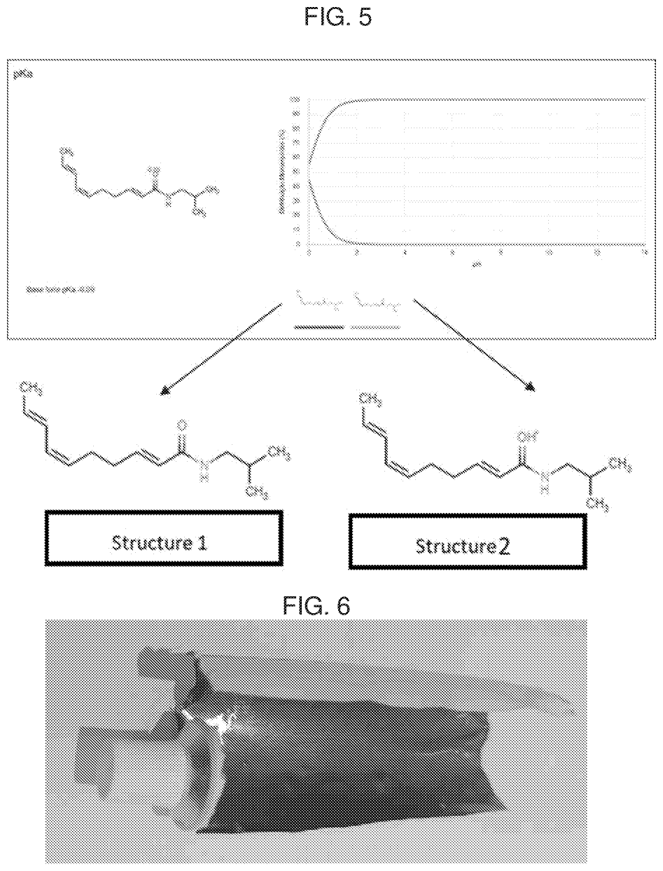

After defining the concentration of the viscosity agent, the development phase itself began through the first prototypes with a focus on one of the main critical quality attributes, which is the pH range, since the predominant degradation kinetics in this product is hydrolysis and the vaginal route of administration. Vaginal pH is approximately pH 3,5 to pH 4,5, (Machado, Preclinical performance of vaginal semisolid products: technological and safety evaluations assuming physiologic parameters. Thesis for Doctoral Degree in Pharmaceutical Sciences. Universidade da Beira Interior, Portugal, 2018), and the product must not cause alterations in this physiological range, therefore, it must have the same pH range. In the pKa graph of spilanthol (FIG. 5) we can identify the predominant chemical forms versus the different pH ranges, spilanthol at pH below 3 is more susceptible to hydrolysis.

The chemical form of the spilanthol molecule that predominates at physiological pH is structure 1, that is, the non-ionized form and less susceptible to degradation and, therefore, the most stable. The formulation, as it has an aqueous base, requires preservatives for microbiological preservation. All preservatives used in pharmaceutical products act in optimal pH ranges and have an intrinsic toxicity due to the function they perform. Sodium benzoate was chosen because it acts at an acidic pH and is less toxic than the vast majority of preservatives, in addition to being used in low and safe doses.

EFSA (2015) classifies spilanthol as class III, with an exposure limit of 90 mg/person/day (60 kg). As the product is for topical use, the defined dose of spilanthol is a maximum of 0.2% (2 mg/g), that is, a maximum of 2% of a standardized plant extract containing 10% spilanthol.

The kinetic potential of degradation by hydrolysis and subsequent oxidation presented by spilanthol is directly related to the pH. In the defined formulation, spilanthol should not undergo significant changes throughout the stability study due to the selected pH range.

The formulation contains a buffering system fixing the pH in the desired range and preventing significant change. The defined buffer system uses citric acid and sodium citrate to obtain a target pH of 4,5, according to the United States Pharmacopoeia—USP (2021). Both have buffering, acidifying and also antioxidant properties, thus promoting greater stability potential for the product. The joint use of lactic acid aimed to keep the vaginal flora balanced, avoiding secondary infections (Plummer, PLoS ONE 16(2): e0246953, 2021). It is a safe excipient at the doses used.

For a synergistic effect of controlling oxidation risks, the excipient disodium edetate (EDTA) was used as a complexing agent to control risks of residual metals, which can occur during manufacturing processes that use stainless steel equipment. Metals can catalyze oxidation reactions and affect the stability of the spilanthol active.

The complete formulation with the active and its excipients resulting from the risk assessments are shown in Table 3. This formulation was named Prototype 1. The first prototypes were manufactured on a laboratory bench scale with 500 g per lot, already with the formulation of Prototype 1. The objective was to preliminarily evaluate the formulation, its properties, and do any adjustments to the physical and sensory characteristics of the product.

TABLE 3

| Components | % |
|---|---|
| Jambu extract (10% Spilanthol) | 2.00 |
| Sodium benzoate | 0.20 |
| Citric acid | 0.80 |
| Sodium citrate | 0.80 |
| Hydroxyethylcellulose | 1.25 |
| Lactic acid | 0.30 |
| Disodium edetate | 0.10 |
| Purified water | 94.55 |

The buffer system using citric acid and sodium citrate was challenged with 1N hydrochloric acid and 1N sodium hydroxide to assess resistance to significant pH changes. It was verified that the robustness of the formulation, facing potential changes in pH, still needed to be optimized, with a proportional increase in the concentration of citric acid and sodium citrate.

Jambu is considered safe (GRAS #3783) by FEMA and EFSA as a food additive, in addition to being traditionally used in food in northern Brazil. Taking into account the safety of Jambu, the team of researchers carried out a sensory evaluation of the oral mucosa for a preliminary assessment of the product in relation to its organoleptic characteristics and market potential. The oral mucosa was used because it is a model close to its application and therefore it was considered that there would be no risks in a laboratory evaluation given the safety of its use in food. The possible contact of the product with the oral mucosa during sexual intercourse (oral sex) was also evaluated. Thus, the effect and sensation on the oral mucosa were also considered relevant for the product both in the sense of its acceptance by the user, and as an indirect way of appreciating its organoleptic characteristics by analogy.

The product tested showed a very intense effect at the previously defined concentration of 2%. Through a risk assessment, the conclusion was that the probability of reports of discomfort in the clinical trial phase in the future would be high. Furthermore, it was considered that the concentration of 2% would be within the maximum limit defined by the EFSA. It was also considered that the 1% dose could present more commercial viability for the product, by making it more accessible to the consumer, in addition to flexibility for eventual line extensions, since for higher concentrations the effect will be known to be greater. Thus, the dose of 10% spilanthol plant extract was adjusted to 1% in the formula for a new sensory evaluation. The conclusion was that this sensory effect was more satisfying (the effect was still present) and pleasant.

The adjusted formulation, called Prototype 2, is presented in Table 4, below.

TABLE 4

| Components | % |
|---|---|
| Jambu extract (10% Spilanthol) | 1.00 |
| Sodium benzoate | 0.20 |
| Citric acid | 0.80 |
| Sodium citrate | 1.60 |
| Hydroxyethylcellulose | 2.50 |
| Lactic acid | 0.30 |
| Disodium edetate | 0.10 |
| Purified water | 93.50 |

Prototype 2 was defined as the ideal candidate to start evaluating primary packaging, prior to stability studies. Transparent PET bottles with pump valve (used in cosmetic products) were tested. During a 30-day stress study, this package did not present adequate airtightness, favoring the loss and evaporation of water, causing an increase in viscosity, in addition to a dry appearance of the gel. An important factor considered was also the high cost of the valves, and the need for filling machines different from those used in the pharmaceutical industry, as “pump” valves are more used in the cosmetics industry, which would restrict the possibility of filling in pharmaceutical laboratories.

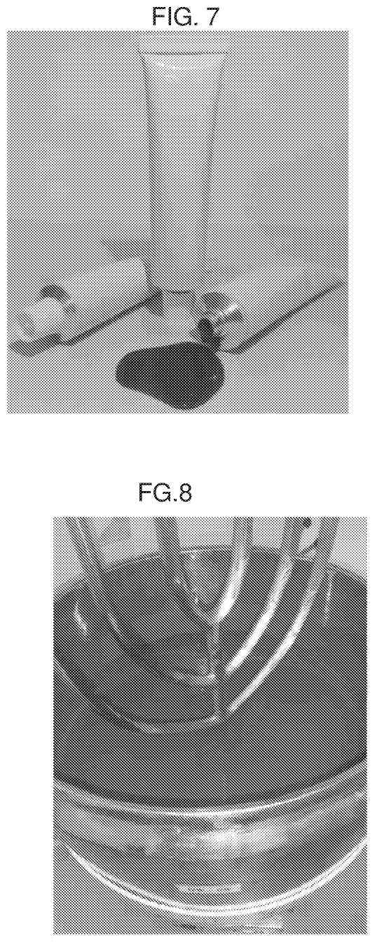

It was defined that aluminum tubes with gold varnish (without titanium dioxide) would be used as an internal coating and with Darex®, a silicone sealant to prevent leaks from the bottom of the tube, present in the sealing region, as shown in FIG. 6, since proven to be non-toxic and harmless to most medicated ointments and creams on the market, reducing the risk of failure due to incompatibilities in confirmation stability studies and in clinical studies to be carried out in the future. In this way, the gel was filled into aluminum tubes (FIG. 7).

The proposed specifications for the finished product (Prototype 2) are in Table 5.

TABLE 5

| Quality Attributes (QA) | Acceptance Criteria | Method |
|---|---|---|
| Aspect (appearance, viscosity, | Green colored gel with characteristic | Visual/Sensory |
| consistency, color, and odor) | odor | |
| pH | Informative | Korolkovas, 1988 |
| | Target - 3.8-4.5 | |
| Determination of Spilanthol | Informative | Santos, 2010 |
| Content by HPLC | Target - 0.05%-0.15% | |
| CCD Chromatographic Profile | Sample: The sample must present, | Bladt, 1996 |
| | in increasing order of Rf | |
| | and below the band | |
| | violet to blue-gray color of weak | |
| | intensity, a band of coloration | |
| | red to brownish color of weak | |
| | intensity and two bands of blue | |
| | color | |
| | weak grayish | |
| Mesophilic Bacteria Count | <100 UFC/g | Farm. Bras. VI |
| Mold and Yeast Count | <100 UFC/g | Farm. Bras. VI |
| Staphylococcus aureus research | Absence/g | Farm. Bras. VI |
| Pseudomonas aeruginosa | Absence/g | Farm. Bras. VI |
| research | | |
| Candida albicans research | Absence/g | Farm. Bras. VI |

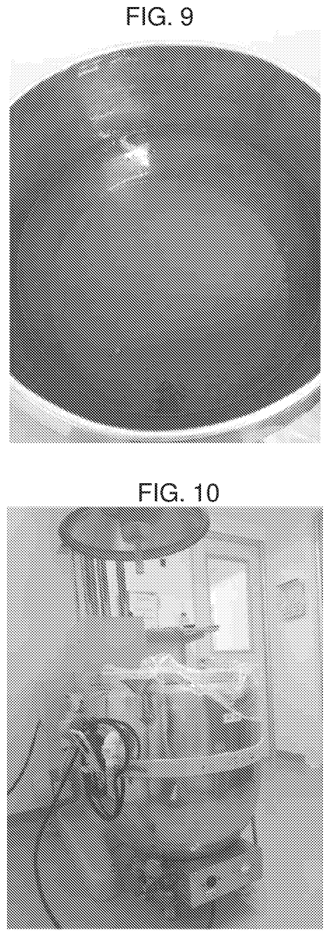

With the definition of the best candidate for stability studies (Prototype 2), the pilot batches were produced at the company Brasterápica Indústria Farmacêutica, as the study batches must be manufactured in industrial equipment (FIGS. 8, 9 and 10), and this company has equipment with capacity for relatively small industrial batches of at least 90 kg, adequate size to obtain a sufficient number of samples for stability studies. The total useful capacity of the equipment was used, which meets the requirements for submission batches for the purposes of registration with the national regulatory agency in accordance with RDC 31/2010 (Anvisa, 2010).

The manufacture of industrial pilot batches was preceded by risk analysis to transfer the scale from bench to industrial, using the FMEA tool, ICH Q9 (ICH, 2005), where the risks were low due to the detection controls used, as per Table 6, below. Risks are ranked according to their RPN (Risk Priority Number) score as: low-green (1 to 99); moderate-yellow (100 to 500); and high-red (501 to 1000). The scores according to severity and criteria were: Barely Perceivable: Score 1 (Does not compromise the patient's health, does not negatively impact the environment. Does not impact customer confidence, and confidence in the product's performance); Little Importance: Score 2, 3 (Dissatisfaction, health impairment, impact on patient confidence); Moderately Severe: Score 4, 5, 6 (Probable loss of treatment efficiency, likely dissatisfaction); Severe: Score 7, 8 (Causes reversible damage to the patient's health, severe negative impact on the environment); and Extremely Severe: Score 9, 10 (Causes irreversible damage to the health or death of the patient, violates legislation, very severe impact on the environment). Regarding the detection rate, and the respective scores and criteria used were: Very High Detection: Score 1 (Very high chance that the controls will detect the cause and subsequent failure); High Detection: Score 2, 3 (High chance that the controls will detect the cause and subsequent failure); Moderate Detection: Score 4, 5, 6 (Moderate chance that the controls will detect the cause and subsequent failure); Low Detection: Score 7, 8 (Low chance that controls will detect the cause and subsequent failure); Remote Sensing: Score 9, 10 (Controls are very poor or inappropriate for detection of cause and subsequent failure, or no controls identified). Risks were based on the following scores: Low: 1 to 99; Moderate: 100 to 500; High: 501 to 1000.

TABLE 6

| | | | | Se- | Oc- | De- | Punc- | |
|---|---|---|---|---|---|---|---|---|
| Process | Failure | Effect of | Existing | ver- | cur- | tec- | tua- | Classi- |
| step | mode | failure | Controls | ity | cene | tion | tion | fication |
| Weighing | Microbi- | Micro- | Process time | 8 | 4 | 2 | 64 | Low |
| of inputs | ological | bial | records in | | | | | |
| | contami- | count | the produc- | | | | | |
| | nation by | out of | tion order | | | | | |
| | exposure | specifi- | | | | | | |
| | | cation | | | | | | |
| | Humidity | For- | Time limita- | 3 | 4 | 3 | 36 | Low |
| | absorp- | mation | tion be- | | | | | |
| | tion | of lumps | tween pro- | | | | | |
| | | and | cess steps | | | | | |
| | | stones in | | | | | | |
| | | the in- | | | | | | |
| | | puts | | | | | | |
| | Incorrect | Content | Double | 9 | 2 | 3 | 54 | Low |
| | weighing | out of | weighing | | | | | |
| | | specifi- | check | | | | | |
| | | cation | | | | | | |
| Addition | Amount | Excess of | The equip- | 7 | 5 | 2 | 70 | Low |
| of Purified | above | water, | ment has | | | | | |
| Water | recom- | product | calibrated | | | | | |
| | mended | dilution | marks | | | | | |
| | Quantity | Difficulty | The equip- | 7 | 5 | 2 | 70 | Low |
| | below | in solu- | ment has | | | | | |
| | recom- | bilizing | calibrated | | | | | |
| | mended | salts | marks | | | | | |
| Addition | Poor sol- | Pres- | Presence of | 8 | 3 | 1 | 24 | Low |
| of Sodium | ubiliza- | ence of | suspended | | | | | |
| Benzoate | tion | sus- | particles, | | | | | |
| and Diso- | | pended | sandy feel- | | | | | |
| dium Ede- | | parti- | ing in the | | | | | |
| tate | | cles, | product | | | | | |
| | | sandy | | | | | | |
| | | feeling | | | | | | |
| | | in the | | | | | | |
| | | product | | | | | | |
| | | Microbi- | This step will | 9 | 3 | 2 | 54 | Low |
| | | ological | be carried | | | | | |
| | | contami- | out in a | | | | | |
| | | nation | smaller pan | | | | | |
| | | defi- | allowing the | | | | | |
| | | ciency in | visualization | | | | | |
| | | preserv- | of the parti- | | | | | |
| | | ative sol- | cles and | | | | | |
| | | ubiliza- | then it will | | | | | |
| | | tion | be added to | | | | | |
| | | | the mixer. | | | | | |
| Stirring | Excessive | Microbi- | Time limita- | 6 | 4 | 1 | 24 | Low |
| until solu- | stirring | ological | tion be- | | | | | |
| bilization | | contami- | tween pro- | | | | | |
| | | nation | cess steps | | | | | |
| | | by ex- | | | | | | |
| | | tending | | | | | | |
| | | the pro- | | | | | | |
| | | cess | | | | | | |
| | | time | | | | | | |
| | Stirring | Pres- | Visual verifi- | 8 | 4 | 2 | 64 | Low |
| | defi- | ence of | cation of sol- | | | | | |
| | ciency | sus- | ubilization | | | | | |
| | | pended | before pro- | | | | | |
| | | parti- | ceeding to | | | | | |
| | | cles, | subsequent | | | | | |
| | | sandy | steps | | | | | |
| | | feeling | | | | | | |
| | | in the | | | | | | |
| | | product | | | | | | |
| Buffer Sys- | Poor sol- | Pres- | Visual verifi- | | | | | Low |
| tem-Cit- | ubiliza- | ence of | cation of sol- | | | | | |
| ric Acid + | tion | sus- | ubilization | | | | | |
| Sodium | | pended | before pro- | | | | | |
| Citrate | | parti- | ceeding to | | | | | |
| | | cles, | subsequent | | | | | |
| | | sandy | steps | | | | | |
| | | feeling | | | | | | |
| | | in the | | | | | | |
| | | product | | | | | | |
| | Buffer | Degra- | Visual verifi- | 9 | 3 | 2 | 54 | Low |
| | not | dation of | cation of sol- | | | | | |
| | formed | the | ubilization | | | | | |
| | | spilan- | before pro- | | | | | |
| | | thol ac- | ceeding to | | | | | |
| | | tive | subsequent | | | | | |
| | | | steps | | | | | |
| | incorrect | Degra- | Visual verifi- | 9 | 2 | 2 | 36 | Low |
| | pH | dation of | cation of sol- | | | | | |
| | | the | ubilization | | | | | |
| | | spilan- | before pro- | | | | | |
| | | thol ac- | ceeding to | | | | | |
| | | tive | subsequent | | | | | |
| | | | steps | | | | | |
| PH verifi- | incorrect | Degra- | pHmeter cal- | 9 | 2 | 1 | 18 | Low |
| cation | pH | dation of | ibrated be- | | | | | |
| | | the | fore use | | | | | |
| | | spilan- | | | | | | |
| | | thol ac- | | | | | | |
| | | tive | | | | | | |
| Addition | For- | Bad as- | Standardiza- | 9 | 5 | 1 | 45 | Low |
| of Hydrox- | mation | pect | tion of the | | | | | |
| yethyl- | of lumps | | form and | | | | | |
| cellulose | | | time of addi- | | | | | |
| | | | tion of Hy- | | | | | |
| | | | droxyethyl- | | | | | |
| | | | cellulose | | | | | |
| | Lack of | Not | Standardiza- | 7 | 5 | 1 | 35 | Low |
| | gelling | proper | tion of the | | | | | |
| | hydra- | gel for- | form and | | | | | |
| | tion | mation | time of addi- | | | | | |
| | | | tion of Hy- | | | | | |
| | | | droxyethyl- | | | | | |
| | | | cellulose; | | | | | |
| | | | Visual check | | | | | |
| | | | before pro- | | | | | |
| | | | ceeding with | | | | | |
| | | | the process | | | | | |
| | Bubble | Weight | Standardiza- | 7 | 6 | 2 | 84 | Low |
| | for- | variation | tion of the | | | | | |
| | mation | when | form and | | | | | |
| | | filling in | time of addi- | | | | | |
| | | tubes | tion of Hy- | | | | | |
| | | | droxyethyl- | | | | | |
| | | | cellulose; | | | | | |
| | | | Control stir- | | | | | |
| | | | ring speed to | | | | | |
| | | | prevent bub- | | | | | |
| | | | bles | | | | | |
| Formation | Excessive | Weight | Standardiza- | 7 | 6 | 3 | 126 | Me- |
| of gel | bubbles | variation | tion of the | | | | | dium |
| | | when | form and | | | | | |
| | | filling in | time of addi- | | | | | |
| | | tubes | tion of Hy- | | | | | |
| | | | droxyethyl- | | | | | |
| | | | cellulose; | | | | | |
| | | | Control stir- | | | | | |
| | | | ring speed to | | | | | |
| | | | prevent bub- | | | | | |
| | | | bles | | | | | |
| Addition | Lack of | Content | Evaluating | 9 | 2 | 3 | 54 | Low |
| of Lactic | homoge- | out of | the mixing | | | | | |
| Acid and | neity | specifi- | time, as the | | | | | |
| Jambu Ex- | | cation | extract is | | | | | |
| tract | | | green in | | | | | |
| | | | color, will | | | | | |
| | | | help to visu- | | | | | |
| | | | ally verify | | | | | |
| | | | the homoge- | | | | | |
| | | | nization po- | | | | | |
| | | | tential. | | | | | |
| | incorrect | pH out | Evaluating | 9 | 2 | 2 | 36 | Low |
| | pH | of speci- | the mixing | | | | | |
| | | fication | time, as the | | | | | |
| | | | extract is | | | | | |
| | | | green in | | | | | |
| | | | color, will | | | | | |
| | | | help to visu- | | | | | |
| | | | ally verify | | | | | |
| | | | the homoge- | | | | | |
| | | | nization po- | | | | | |
| | | | tential. | | | | | |
| | | Degra- | Evaluating | 9 | 2 | 4 | 72 | Low |
| | | dation of | the mixing | | | | | |
| | | the | time, as the | | | | | |
| | | spilan- | extract is | | | | | |
| | | thol ac- | green in | | | | | |
| | | tive | color, will | | | | | |
| | | | help to visu- | | | | | |
| | | | ally verify | | | | | |
| | | | the homoge- | | | | | |
| | | | nization po- | | | | | |
| | | | tential. | | | | | |
| PH verifi- | incorrect | Degra- | pHmeter cal- | 9 | 2 | 2 | 36 | Low |
| cation | pH | dation of | ibrated be- | | | | | |
| | | the | fore use | | | | | |
| | | spilan- | | | | | | |
| | | thol ac- | | | | | | |
| | | tive | | | | | | |
| Volume | Excessive | Content | Qualified | 9 | 2 | 3 | 54 | Low |
| adjust- | purified | out of | equipment | | | | | |
| ment with | water | specifi- | with cali- | | | | | |
| Purified | | cation | | | | | | |
| Water | Purified | Content | brated | 9 | 2 | 1 | 18 | Low |
| | water | out of | marks | | | | | |
| | defi- | specifi- | | | | | | |
| | ciency | cation | | | | | | |
| | Excessive | Microbi- | Time limita- | 9 | 2 | 1 | 18 | Low |
| | process | ological | tion be- | | | | | |
| | time | contami- | tween pro- | | | | | |
| | | nation | cess steps | | | | | |
| | | | and record- | | | | | |
| | | | ing of the | | | | | |
| | | | start and | | | | | |
| | | | end of the | | | | | |
| | | | step | | | | | |
| Filling in | Average | Loss of | Evaluate | 6 | 4 | 1 | 24 | Low |
| aluminum | Weight | product, | processing | | | | | |
| tubes with | Variation | impaired | capacity | | | | | |
| gold and | | yield | (cpk > 1,33) | | | | | |
| darex var- | | | versus | | | | | |
| nish | | | evader | | | | | |
| | | | speed. | | | | | |

Still according to RDC 31/2010 (Anvisa, 2010), three batches of 90 kg each were manufactured, for accelerated and long-term stability studies, and also for transferring the process from bench scale to industrial scale, defining the parameters and final adjustments of development of the manufacturing process.

The analytical developments and the respective validations of the methodologies were carried out for the analysis of the active spilanthol in the raw material and in the finished product, using a service provider laboratory certified as Reblas by Anvisa (2020) for analytical services for phytotherapeutic inputs and products.

The three batches manufactured in industrial equipment were sent for quality control analysis and the results were considered initial reference (T0) for stability studies (Table 7).

TABLE 7

| Quality Attributes | Acceptance criteria | | | |
|---|---|---|---|---|
| (QA) | (preliminary specification) | Batch 1 | Batch 2 | Batch 3 |
| Aspect (appearance, | Green colored gel with | In conformity | In conformity | In conformity |
| viscosity, | characteristic odor | | | |
| consistency, color, | | | | |
| and odor) | | | | |
| pH | Informative | 4.46 | 4.42 | 4.40 |
| | Target - 3.8-4.5 | | | |
| Determination of | Informative | 0.13% | 0.13% | 0.13% |
| Spilanthol Content | Target - 0.05%-0.15% | | | |
| by HPLC | | | | |
| CCD Chromatographic | Sample: The sample must | In conformity | In conformity | In conformity |
| Profile | present, in increasing order | | | |
| | of Rf and below the violet to | | | |
| | grayish-blue color band of | | | |
| | weak intensity, a color band | | | |
| | red to brownish color of | | | |
| | weak intensity and two | | | |
| | bands of blue color | | | |
| | weak grayish | | | |
| Mesophilic Bacteria | <100 UFC/g | <10 UFC/g | <10 UFC/g | <10 UFC/g |
| Count | | | | |
| Mold and Yeast | <100 UFC/g | <10 UFC/g | <10 UFC/g | <10 UFC/g |
| Count | | | | |
| Staphylococcus | Absence/g | Absent | Absent | Absent |
| aureus research | | | | |
| Pseudomonas | Absence/g | Absent | Absent | Absent |
| aeruginosa research | | | | |
| Candida albicans | Absence/g | Absent | Absent | Absent |
| research | | | | |

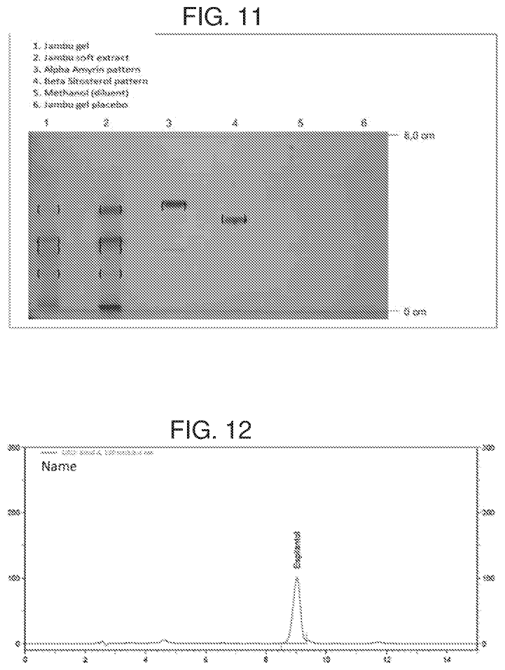

In accordance with the specification shown in Table 5, the phytochemical profile of the jambu gel sample showed, in increasing order of Rf and below the alpha amyrin standard band, a violet to grayish-blue color band of weak intensity, a band of red to brownish color of weak intensity and two bands of grayish blue color of weak intensity as can be seen in FIG. 11.

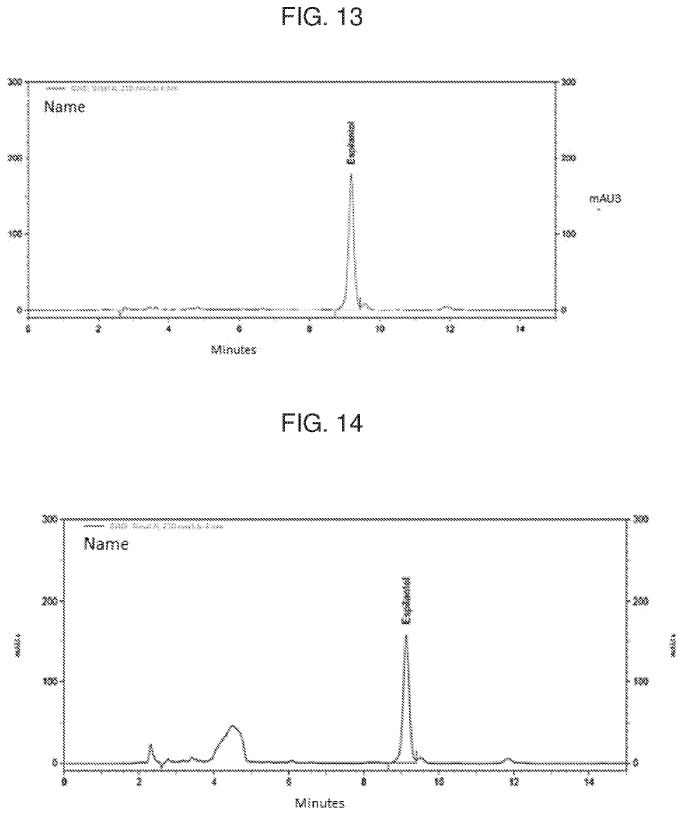

Chromatographic profiles by HPLC showed good resolution and relatively short retention times (approximately 9 minutes), enabling the analysis times in quality control, and without interference peaks, not only for the spilanthol standard but also for the finished product (jambu gel), as shown in FIGS. 12 and 14. The excipients of the jambu extract also showed a chromatogram without interference, as shown in FIG. 13.

The analytical development for spilanthol content was an adaptation based on Santos (2010) for the plant extract, which required optimizations and adaptations for testing the finished product, considering the formulation with the base and combination of inputs used.

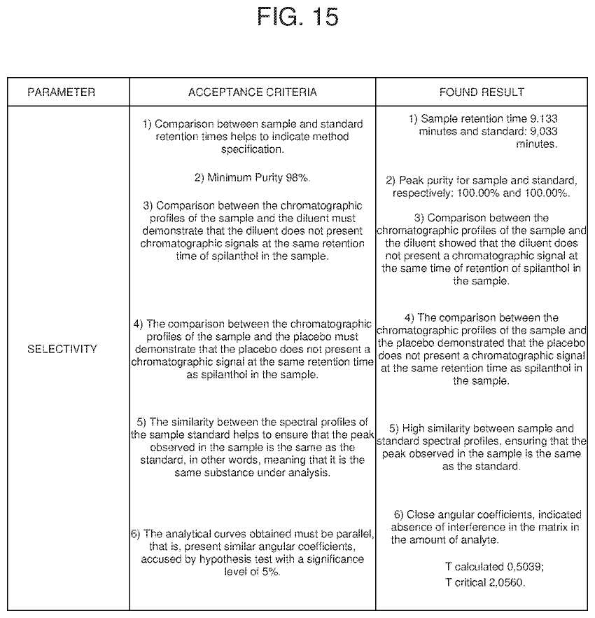

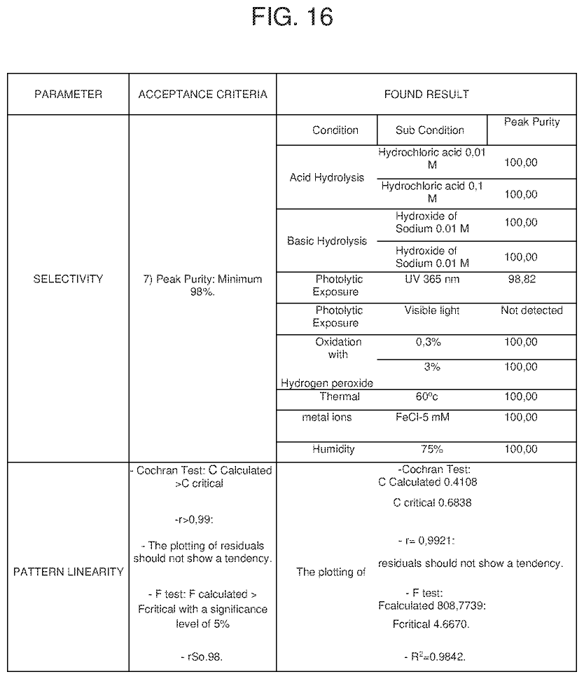

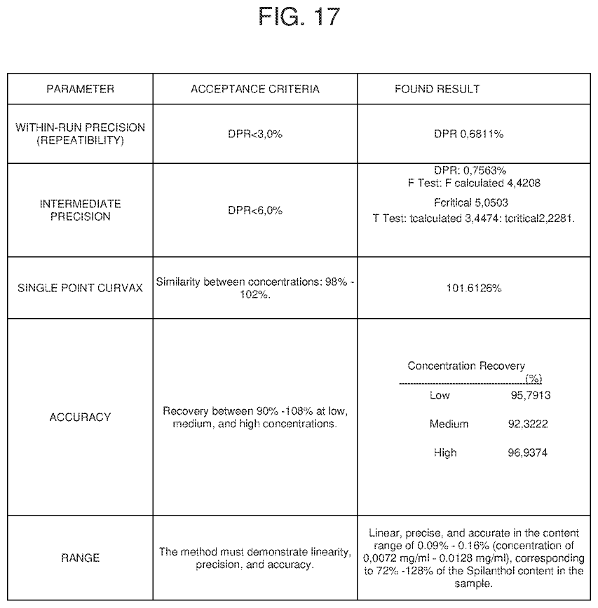

The methods proved to be selective (indicative of stability), robust, precise and accurate, confirming their applicability according to the results of the tests carried out and reproduced in FIGS. 15, 16, and 17.

The results for the accelerated stability study tests for lot 1, 2, and 3 are shown in Table 8.

TABLE 8

| Quality Attributes | | | |
|---|---|---|---|
| (QA) | Acceptance Criteria | Initial | 3 months |
| Aspect (appearance, | Green colored gel with characteristic | In conformity | In conformity (shade |
| viscosity, consistency, | odor | | slightly lighter than |
| color, and odor) | | | at time zero) |
| pH | Informative | 4.46 | 4.33-4.34 |
| | Target - 3.8-4.5 | | |
| Determination of | Informative | 0.13% | 0.08% |
| Spilanthol Content by | Target - 0.05%-0.15% | | |
| HPLC | | | |
| CCD Chromatographic | Sample: The sample must present, | In conformity | In conformity |
| Profile | in increasing order of Rf and below | | |
| | the band | | |
| | violet to blue-gray color of weak | | |
| | intensity, a band of coloration | | |
| | red to brownish color of weak | | |
| | intensity and two bands of blue color | | |
| | weak grayish | | |
| Mesophilic Bacteria | <100 UFC/g | <10 UFC/g | <10 UFC/g |
| Count | | | |
| Mold and Yeast Count | <100 UFC/g | <10 UFC/g | <10 UFC/g |
| Staphylococcus aureus | Absence/g | Absent | Absent |
| research | | | |
| Pseudomonas aeruginosa | Absence/g | Absent | Absent |
| research | | | |
| Candida albicans research | Absence/g | Absent | Absent |

The 3-month results of the accelerated stability studies are within specifications. They showed an apparent reduction in content that will be analyzed together with the 6-month results. The conditions of the accelerated study are at 40° C. +/−2° C. and 75% +/−5% of variation, being a very stressful condition for the product, according to RDC 318/2019 (Anvisa, 2019). It is important to emphasize that this is a non-decisive stress test and that the indication of stability is only defined by the long-term study, according to the ICH Q1A (ICH, Requirements for Registration of Pharmaceuticals for Human Use. Evaluation for stability data Q1E, 2003). The accelerated stability study provides security and a provisional validity period if the registration is submitted before the end of the long-term study, RDC 318/2019 (Anvisa, 2019).

The results for the long-term stability study tests for lot 1, 2, and 3 are shown in Table 9.

TABLE 9

| Quality Attributes (QA) | Acceptance Criteria | Initial | 3 months |
|---|---|---|---|
| Aspect (appearance, | Green colored gel with characteristic | In conformity | In conformity |
| viscosity, consistency, | odor | | |
| color, and odor) | | | |
| pH | Informative | 4.40-4.42 | 4.36-4.40 |
| | Target - 3.8-4.5 | | |
| Determination of | Informative | 0.13% | 0.12% |
| Spilanthol Content by | Target - 0.05%-0.15% | | |
| HPLC | | | |
| CCD Chromatographic | Sample: The sample must present, in | In conformity | In conformity |
| Profile | increasing order of Rf and below the | | |
| | band | | |
| | violet to blue-gray color of weak | | |
| | intensity, a band of coloration | | |
| | red to brownish color of weak | | |
| | intensity and two bands of blue color | | |
| | weak grayish | | |
| Mesophilic Bacteria | <100 UFC/g | <10 UFC/g | <10 UFC/g |
| Count | | | |
| Mold and Yeast Count | <100 UFC/g | <10 UFC/g | <10 UFC/g |
| Staphylococcus aureus | Absence/g | Absent | Absent |
| research | | | |
| Pseudomonas aeruginosa | Absence/g | Absent | Absent |
| research | | | |
| Candida albicans research | Absence/g | Absent | Absent |

The results of the 3-month long-term stability studies remain unchanged compared to the initial results, demonstrating that it remains stable, with no degradation of the active ingredient to this date.

An innovative product was developed for use as an adjuvant in the treatment of sexual dysfunctions using concentrated jambu (Spilanthes acmella var oleracea) extract. A stable formulation was obtained in terms of physical, chemical, and microbiological aspects, compatible with industrially viable application on the vaginal and anal mucosa, and in accordance with current regulations and guidelines.

The research carried out adequacy in the planned coloring and concentration of spilanthol. The first for not using the excipient titanium dioxide as a bleaching agent, due to the recent questions that are taking place regarding the safety of its use and for the gel obtained, which is green in color and has a translucent appearance, which must meet the demand of the modern consumer for products with an attractive natural appearance and free of additives. The second adaptation was due to the results of the evaluation of the organoleptic characteristics, which indicated that the 1% concentration of spilanthol was safer and more adequate.

The analytical test methods were developed and the respective validations carried out following the specific legislation and with compliant results for the formulation developed in this project. The technical adequacy tests of the formulation quality and the accelerated and long-term stability studies showed satisfactory results, and none of them were out of specifications. Thus, it is concluded that the developed product meets the quality and stability requirements required by Anvisa for registration purposes.

The formulations of the present invention can be used in medicines and cosmetics for topical use in both the genital and anal areas. It is interesting to point out that the formulations of the present invention can be used in any of the phases of female and male sexual life, helping with sexual dysfunctions present in different stages of development, from the beginning of sexual life to menopause or andropause.

The present invention is not restricted to the representations described herein and the illustrations shown. Changes or replacements of components within the scope of the invention can be considered as elements of the presented invention. The formulations shown here are examples, other forms and modifications being considered as included in the scope of the claims. The terms adopted here are not intended to act as limitations, on the contrary, they should be understood in a generic way.

1-8. (canceled)

9. A phytotherapy formulation for preventing or treating sexual dysfunctions comprising:
(a) a standardized extract of aerial parts of Spilanthes acmella containing from 1% to 10% of spilanthol marker;
(b) sodium benzoate;
(c) citric acid;
(d) sodium citrate;
(e) hydroxyethylcellulose,
(f) lactic acid;
(g) disodium edetate, and
(h) purified water.

10. The phytotherapy formulation according to claim 9, wherein the spilanthol is present in the range of 0.1 mg/g to 3 mg/g,

11. The phytotherapy formulation according to claim 9, wherein the spilanthol is in the range of 0.5 mg/g to 2.5 mg/g.

12. The phytotherapy formulation according to claim 9, having a pH in the range of 3.8 to 4.5.

13. The phytotherapy formulation according to claim 9, wherein the formulation is a gel, cream, or ointment.

14. A method of treating female and male sexual dysfunctions selected from the group consisting of hypoactive sexual desire disorder (HSDD); vaginal dryness; and pain during vulvar anal sexual intercourse, said method comprising administering the standardized extract of aerial parts of Spilanthes acmella according to claim 9.

15. A cosmetic or health product for vulvar or anal topical administration comprising the phytotherapy formulation according to claim 9.

16. A method of treating female and male sexual dysfunctions selected from the group consisting of hypoactive sexual desire disorder (HSDD); vaginal dryness; and pain during vulvar or anal sexual intercourse; said method comprising administering the standardized extract of aerial parts of Spilanthes acmella containing from 1% to 10% of spilanthol marker.

17. A cosmetic or health product for vulvar or anal topical administration comprising a standardized extract of aerial parts of Spilanthes acmella containing from 1% to 10% of spilanthol marker.
